(19) United States
(12) Patent Application Publication
(10) Pub. No.: US 2022/0288081 A1
(43) Pub. Date: Sep. 15, 2022

(54) TREATMENT OF EXCITOTOXICITY-RELATED CONDITIONS

(71) Applicant: Macquarie University, North Ryde (AU)

(72) Inventors: Lars Ittner, North Ryde (AU); Yazi Diana Ke, North Ryde (AU)

(21) Appl. No.: 17/754,134

(22) PCT Filed: Sep. 25, 2020
(86) PCT No.: PCT/AU2020/051023

§ 371 Date: Mar. 24, 2022

(30) Foreign Application Priority Data
Sep. 25, 2019 (AU) 2019 903588

(51) Int. Cl. A61K 31/519; A61P 25/08

(52) U.S. Cl. CPC A61K 31/519; A61P 25/08

(57) ABSTRACT

Disclosed herein are methods for treating or preventing an excitotoxicity-related condition, optionally a condition associated with seizures and/or resulting from or associated with a cerebral ischemic event, comprising administering to a subject in need an effective amount of an inhibitor of Lim-domain kinase 1 (LIMK1). Also provided are methods for treating or preventing seizures and for reducing excitotoxicity in neurons and/or protecting neurons from excitotoxicity.

### FIELD OF THE ART

The present disclosure relates generally to methods for reducing excitotoxicity and for the treatment and prevention of excitotoxicity-associated conditions such as epilepsy and seizures resulting from cerebral ischemic events.

### BACKGROUND

Glutamate is one of the major excitatory neurotransmitters widely expressed in the brain. N-methyl-D-aspartate (NMDA) receptors are a group of glutamate receptor that mediate excitatory synaptic transmission in the brain, playing a key role in excitotoxicity, synaptic plasticity, synaptogenesis, memory acquisition and learning. Excitotoxicity, a pathophysiological process characterized by neuronal overexcitation and resulting in cellular and/or neuronal network dysfunction or death, is a principal cause of seizures and is associated with a variety or neurological conditions and ischemic events. Seizures may or may not be accompanied by loss of awareness or loss of consciousness. Seizures are typically classified as being focal, resulting from abnormal electrical activity in one region of the brain, or generalized, resulting from abnormal electrical activity in multiple regions of the brain. Generalized seizures can also be classified by reference to the clinical manifestation of the seizure, including absence seizures (petit mal seizures characterized by absent expression and subtle body movements), tonic seizures (muscle stiffening), atonic seizures (loss of muscle control), clonic seizures (repeated jerking muscle movements), myoclonic seizures (sudden jerks or twitches) and tonic-clonic seizures (grand mal seizures characterized by body stiffening, shaking, loss of consciousness, possible loss of bladder control and/or biting of the tongue).

Seizures are a common manifestation of neuronal overexcitation, and epilepsy is one of the more prevalent neurological conditions, estimated to affect between 1-5% of the population. Epilepsy is characterized by a tendency to recurrent seizures that can lead to loss of awareness, loss of consciousness, and/or disturbances of movement, typically tonic-clonic (grand mal) seizures. The primary pathology of an epileptic seizure is an abnormal hypersynchronization of electrical activity between large numbers of neurons of the cerebral cortex.

Seizures can also result from cerebral ischemic events such as stroke or traumatic brain injury. Stroke occurs when focal cerebral ischemia is severe and/or prolonged. Stroke is the leading cause of seizures in the elderly population and a proportion of stroke sufferers will go on to develop post-stroke epilepsy. Traumatic brain injury typically occurs blunt force trauma to the head. Seizures can also result from neurodegeneration, such as in Alzheimer's disease. Such seizures may present as increased incidence of overt epilepsy or silent, non-convulsive seizure activity during electroencephalography recording

Current efforts to reduce excitotoxicity have been met with limited success. There is a clear need for the development of new treatments to reduce excitotoxicity and treat conditions associated with excitotoxicity such as epilepsy and post-ischemic seizures.

### SUMMARY OF THE DISCLOSURE

The present disclosure is predicated on the inventors' findings that genetic depletion and pharmacological inhibition of LIM-domain kinase 1 (LIMK1) reduce excitotoxicity of neurons, reduce excitotoxic seizures and protect against excitotoxic seizures in a mouse model.

According to a first aspect of the present disclosure there is provided a method for treating or preventing an excitotoxicity-related condition in a subject, the method comprising administering to the subject an effective amount of an inhibitor of Lim-domain kinase 1 (LIMK1).

Typically the excitotoxicity-related condition is associated with seizures. The condition may result from or be associated with a cerebral ischemic event. By way of example, the cerebral ischemic event may comprise a traumatic brain injury or stroke. The excitotoxicity-related condition may be epilepsy. The seizures or epilepsy may be due to the presence of one or more underlying genetic variants resulting in an epilepsy syndrome or syndrome associated with epilepsy.

Treating or preventing an excitotoxicity-related condition may comprise reducing the severity of a seizure or of seizures over time, increasing the latency to develop a seizure or seizures over time, and/or reducing the frequency of seizures. Treating or preventing an excitotoxicity-related condition may comprise reducing excitotoxicity in neurons and/or for protecting neurons from excitotoxicity.

The inhibitor may be an inhibitor of LIMK1 expression and/or an inhibitor of LIMK1 activity. The inhibitor may be a selective LIMK1 inhibitor. The inhibitor may be a specific LIMK1 inhibitor.

In an exemplary embodiment, the inhibitor may be a small molecule inhibitor.

In an exemplary embodiment, the inhibitor may comprise a compound of Formula (I) or a pharmaceutically acceptable salt thereof:

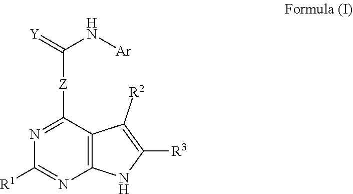

wherein:

Z is selected from the group consisting of optionally substituted cycloalkylene, optionally substituted arylene and optionally substituted aniline;

R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;

Y is selected from the group consisting of O, S, NCN, NCS and NSO₂Me; and

Ar is selected from the group consisting of optionally substituted aryl, optionally substituted heteroaryl and optionally substituted heterocyclyl.

In some embodiments, Z is selected from one of the following structures:

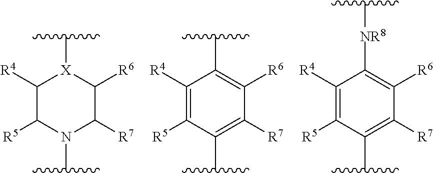

wherein:

R⁴, R⁵, R⁶ and R⁷ have the same definition as R² and R³ above;

X is CH or N; and

R⁸ is H or optionally substituted alkyl.

In a particular embodiment, the compound of Formula (I) is the compound of Formula (Ia) or a pharmaceutically acceptable salt thereof:

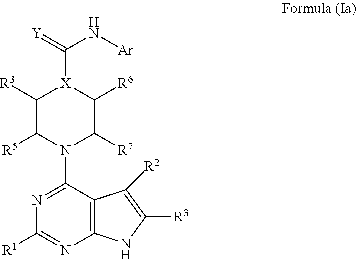

wherein variables R¹ to R⁷, X, Y and Ar are as defined above.

In an embodiment, the compound of Formula (I) is the compound of Formula (Ia), or a pharmaceutically acceptable salt thereof, wherein:

R¹, R³, R⁵, R⁶ and R⁷ are H;

R² is methyl;

R³ is (S)-methyl;

X is N;

Y is NCN; and

Ar is 3-bromophenyl.

In an embodiment, the compound of Formula (Ia) has the following structure:

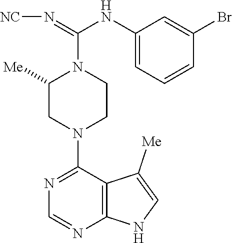 [SMILES: Cc1c[nH]c2ncnc(N3CCN(/C(=N\C#N)Nc4cccc(Br)c4)[C@@H](C)C3)c12]

According to a second aspect of the present disclosure there is provided a method for treating or preventing seizures in a subject in need thereof, the method comprising administering to the subject an effective amount of an inhibitor of LIMK1.

The seizures may be focal or generalized seizures. The seizures may be, for example, absence seizures, tonic seizures, atonic seizures, clonic seizures, myoclonic seizures or tonic-clonic seizures.

In exemplary embodiments, treating or preventing seizures may comprise reducing the severity of a seizure or of seizures over time, increasing the latency to develop a seizure or seizures over time, and/or reducing the frequency of seizures. Treating or preventing seizures may comprise protecting neurons from excitotoxicity.

The subject may have, be susceptible to, or at risk of developing, an excitotoxicity-related condition, such as epilepsy.

In an exemplary embodiment, the inhibitor may comprise a compound of Formula (I) as defined hereinbefore, or a pharmaceutically acceptable salt thereof.

According to a third aspect of the present disclosure there is provided a method for reducing excitotoxicity in neurons and/or for protecting neurons from excitotoxicity, the method comprising exposing neurons to an effective amount of an inhibitor of LIMK1.

Neurons may be exposed to the inhibitor in vivo or ex vivo.

In accordance with the third aspect, the LIMK1 inhibitor may be administered to a subject having, susceptible to, or at risk of developing, an excitotoxicity-related condition or a subject experiencing, susceptible to, or at risk of experiencing, seizures.

In an exemplary embodiment, the inhibitor may comprise a compound of Formula (I) as defined hereinbefore or a pharmaceutically acceptable salt thereof.

A fourth aspect of the present disclosure provides the use of an inhibitor of LIMK1 in the manufacture of a medicament for treating or preventing an excitotoxicity-related condition.

Typically the excitotoxicity-related condition is associated with seizures. The condition may result from or be associated with a cerebral ischemic event. By way of example, the cerebral ischemic event may comprise a traumatic brain injury or stroke. The excitotoxicity-related condition may be epilepsy.

A fifth aspect of the present disclosure provides the use of an inhibitor of LIMK1 in the manufacture of a medicament for treating or preventing seizures.

A sixth aspect of the present disclosure provides the use of an inhibitor of LIMK1 in the manufacture of a medicament for reducing excitotoxicity in neurons and/or for protecting neurons from excitotoxicity.

### BRIEF DESCRIPTION OF THE DRAWINGS

Aspects and embodiments of the present disclosure are described herein, by way of non-limiting example only, with reference to the following drawings.

FIG. 1. Reduced epileptic activity and network hypersynchronisity upon LIMK1 depletion in APP transgenic mice. (A) Left graph: Significantly increased number of spikes per minute in APP transgenic APP23 mice (APP23/Limk1^{+/+}) as compared with non-transgenic (Limk1^{+/+}) and Limk1 knockout (Limk1^{-/-}) littermates during telemetric hippocampal electroencephalography (EEG) recordings in freely moving mice. Spike frequency was significantly lower in EEG recordings of APP23/Limk1^{-/-} mice as compared with APP23/Limk1^{+/+} littermates, and not significantly (ns) different from Limk1^{+/+} and Limk1^{-/-} controls. n=11, *p<0.05, ***p<0.001 (Student's t tests). Right graph: Display of spike frequency in EEG recordings of individual Limk1^{+/+}, Limk1^{-/-}, APP23/Limk1^{+/+} and APP23/Limk1^{-/-} mice statistically analysed in the left graph. (B) Significantly reduced modulation index of cross frequency coupling in APP23/Limk1^{+/+} mice as compared with APP23/Limk1^{-/-} littermates and Limk1^{+/+} and Limk1^{-/-} controls. n=8, ***p<0.001 (Student's t tests) (C) Significantly disrupted amplitude phase in EEG recordings of APP23/Limk1^{+/+} mice as compared with APP23/Limk1^{-/-} littermates and Limk1^{+/+} and Limk1^{-/-} controls. n=8, *p<0.05, **p<0.01 (ANOVA). For (A) and (B), from left to right: Limk1^{+/+}; Limk1^{-/-}; APP23/Limk1^{+/+}; APP23/Limk1^{-/-}.

FIG. 2. Reduced susceptibility to induced seizures in Limk1^{-/-} mice. Excitotoxic seizures were induced by acute intraperitoneal injection of 50 mg/kg body weight pentylenetetrazole (PTZ) followed by timing and scoring (0=no seizures to 7=terminal status epilepticus) of seizure development. Left graph: Similar latency to develop lower grade seizures (i.e. score <5) in Limk1^{-/-} (lower line) mice compared to Limk1^{+/+} littermates. Only Limk1^{+/+} mice develop severe seizure stages over time (i.e. score ≥5). Right graph: Significantly reduced seizure severity in Limk1^{-/-} mice (right hand column) as compared with non-mutant Limk1^{+/+} littermates (left hand column). n=6, *p<0.05 (Student's t test).

FIG. 3. LIMK1 inhibition reduced susceptibility to induced seizures and mitigated neuronal network hypersynchronisity in mice. (A) Excitotoxic seizures were induced by acute intraperitoneal injection of 50 mg/kg body weight pentylenetetrazole (PTZ) followed by timing and scoring (0=no seizures to 7=terminal status epilepticus) of seizure development. Mice were treated with vehicle or 10 mg/kg body weight LIMK1 inhibitor LIMKi intraperitoneally 30 minutes prior to seizure induction. Left graph: Delayed latency to develop lower grade seizures (i.e. score <5) in LIMKi-treated mice (darker line) compared with vehicle controls. Only vehicle-treated mice develop severe seizure stages over time (i.e. score=5). Right graph: Significantly reduced seizure severity in LIMKi-treated mice (right hand column) as compared with vehicle controls (left hand column). n=9, **p<0.001 (Student's t test). (B) Compound hippocampal electroencephalography (EEG) recordings over 4 hours in non-transgenic (wt) and APP transgenic (APP23) mice including 2 hours before (Pre) and 2 hours after (Post) LIMKi or vehicle administration. Time of intraperitoneal LIMKi and vehicle administration is indicated with an arrow. Left: No overt spike activity pre and post vehicle or LIMKi injection in wt mice. Frequent spikes pre and post vehicle administration in APP23 mice. In contrast, LIMKi injection mitigates spike activity in APP23 post injection. Right graphs: No significant changes in spike frequency in vehicle-treated APP23 and significantly reduced spike frequency in LIMKi treated APP23 mice when comparing pre and post spike numbers. n=8, *p<0.05 (Student's t test). (C) Significantly reduced modulation index of cross frequency coupling of APP23 mice (vehicle) was corrected 1 hours after LIMKi administration and no longer different from wt controls (vehicle/LIMKi-treated) n=8, **p<0.01 (Student's t tests). From left to right: non-Tg+vehicle; non-Tg+LIMKi; APP23+vehicle; APP23+LIMKi. (D) Significantly disrupted amplitude phase in EEG recordings of APP23 mice (vehicle) was corrected 1 hour after LIMKi delivery and no longer different from wt controls (vehicle/LIMKi-treated). n=8, *p<0.05 (ANOVA).

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

The term “inhibitor” as used herein refers to an agent that decreases or inhibits at least one function or biological activity of a target molecule, e.g. LIMK1, either directly or indirectly. The term “selective” and grammatical variants thereof are used herein to refer to agents that inhibit a target molecule without substantially inhibiting the function of another molecule.

In the context of the present disclosure, the terms “inhibiting” and grammatical equivalents do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably. Similarly, the terms “inhibit”, “decrease” and “reduce” may be used interchangeably, in reference to the level of, or a value for, a substance, phenomenon, function or activity in a second sample or at a second timepoint that is lower than the level of, or value for, the substance, phenomenon, function or activity in a first sample or at a first timepoint. The reduction may be determined or measured subjectively or objectively, and may be subject to an art-accepted statistical method of analysis.

Use of the term “associated with” herein describes a temporal, physical or spatial relationship between events, symptoms or pathologies. Thus for example, in the context of the present disclosure an excitotoxicity-related condition associated with seizures or associated with a cerebral ischemic event, means that the condition is at least partially characterized by, or results from, either directly or indirectly, the seizures or the cerebral ischemic event. The condition may occur or begin at the time of the ischemic event. Alternatively, the ischemic event and the condition may be temporally spaced such that the onset of the condition is minutes, hours, days, weeks, months or years after the occurrence of the ischemic event.

As used herein the terms “treating”, “treatment”, “preventing”, “prevention” and grammatical equivalents refer to any and all uses which remedy the stated neurodegenerative disease, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein the term “effective amount” includes within its meaning a non-toxic but sufficient amount or dose of an agent or compound to provide the desired effect. The exact amount or dose required will vary from subject to subject depending on factors such as the species being treated, the age, size, weight and general condition of the subject, the severity of the disease or condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The LIM-domain family of protein kinases includes LIM-domain kinase 1 (LIMK1) and LIM-domain kinase 2 (LIMK2). The LIM kinases are serine/threonine protein kinases which bind actin, influencing the architecture of the actin cytoskeleton by regulating the activity of cofilin proteins through phosphorylation. Most highly expressed in the brain, neuronal LIMK1 is an established regulator of synaptic morphology. Prior to the present invention little was known about the functional role of LIMK1 in neurological disease. Human LIMK1 is a 647 amino acid protein (UniProt protein database Accession No. P53667) produced from the LIMK1 gene encoded on chromosome 7. Alternative splicing produces four isoforms of LIMK1.

As exemplified herein, the inventors have demonstrated that genetic depletion of LIMK1 renders mice less susceptible to excitotoxic seizures, and further that pharmacological inhibition of LIMK1 protects neurons from excitotoxicity, reduces the severity of excitotoxic seizures and increases the latency to develop severe seizures.

In one aspect the present disclosure provides a method for treating or preventing an excitotoxicity-related condition in a subject, the method comprising administering to the subject an effective amount of an inhibitor of Lim-domain kinase 1 (LIMK1).

Another aspect of the present disclosure provides a method for treating or preventing seizures in a subject in need thereof, the method comprising administering to the subject an effective amount of an inhibitor of LIMK1. The subject may have, be susceptible to, or at risk of developing, an excitotoxicity-related condition. A further aspect of the disclosure relates to a method for reducing excitotoxicity in neurons and/or for protecting neurons from excitotoxicity, the method comprising exposing neurons to an effective amount of an inhibitor of LIMK1.

Typically, sufferers of an excitotoxicity-related condition experience seizures. The seizures may be focal or generalized seizures, and may be characterised, for example as absence (petit mal) seizures, tonic seizures, atonic seizures, clonic seizures, myoclonic seizures or tonic-clonic (grand mal) seizures. Seizures may also differ in severity. Several seizure severity scales have been developed, including the Chalfont Seizure Severity Scale (Duncan and Sander, 1991, J Neurol Neurosurg Psychiatry 54:873-876) and the National Hospital Seizure Severity Scale (O'Donoghue et al., 1996, Epilepsia 37:563-571). Such scales provide an exemplary means of diagnosing the condition suffered by a subject to be treated in accordance with the present disclosure, and an exemplary means of monitoring progress and outcomes of treatments performed in accordance with the present disclosure. For example, treating an excitotoxicity-related condition or treating or preventing seizures may comprise reducing the severity of a seizure or of multiple seizures over time. Similarly, treating an excitotoxicity-related condition or treating or preventing seizures may comprise increasing the latency to develop a seizure or seizures over time, and/or reducing the frequency of seizures.

Excitotoxicity-related conditions that may be treated or prevented in accordance with the present disclosure include epilepsy and conditions associated with or resulting from a cerebral ischemic event such as an acquired or traumatic brain injury or stroke.

Epilepsy refers to a group of neurological conditions characterized by seizures and the scope of the present disclosure is not limited by reference to any one specific form or type of epilepsy. As noted above, seizures associated with the epilepsy can be focal or generalized and may be characterized as, for example, absence (petit mal) seizures, tonic seizures, atonic seizures, clonic seizures, myoclonic seizures or tonic-clonic (grand mal) seizures. The epilepsy may be hereditary or be acquired (for example resulting from hippocampal sclerosis, perinatal infection, cerebral trauma or infection, stroke, a cerebrovascular disorder, a cerebral immunological disorder or other neurological condition), or alternatively the epilepsy may have no known cause. The epilepsy may be for example, Dravet Syndrome, West Syndrome, Doose Syndrome (myoclonic astatic epilepsy), Rolandic epilepsy, Rasmussen's Syndrome, Lennox-Gastaut Syndrome, Landau-Kleffner Syndrome, Sturge-Weber Syndrome, Otohara Syndrome, Angelman Syndrome, Glut1 Deficiency Syndrome, PCDH19 Epilepsy, a progressive myoclonic epilepsy, a neurocutaneous syndrome, a frontal lobe epilepsy or a juvenile myoclonic or absence epilepsy.

The term acquired brain injury is used to refer to damage to the brain that occurs after birth and is not, of itself, related to a congenital or degenerative condition. Thus, acquired brain injury includes traumatic brain injury, which occurs when the brain sustains damages from a sudden trauma. Traumatic brain injury may comprise mild traumatic brain injury, chronic traumatic encephalopathy, or concussion. The severity of a traumatic brain injury can vary from mild to moderate or severe and symptoms may appear immediately or within days, weeks, months or years after the traumatic event. Symptoms of traumatic brain injury may include headache, confusion, dizziness, changes in mood, and impairment in cognitive function, such as memory, learning, and attention, nausea, convulsions or seizures, slurring of speech, numbness of extremities, and loss of coordination. Traumatic brain injury results from a mild, moderate or severe trauma or injury to the head. Traumatic brain injuries that may result in seizures and excitotoxicity-related conditions include motor vehicle accidents, sports injuries, occupational hazards, physical violence and falls, for example causing a concussion. Examples of traumatic brain injury include motor vehicle accidents, sports injuries, occupational hazards, physical violence and falls. Stroke, hypoxic-ischemia, haemorrhage, encephalitis, and related acquired encephalopathies are other exemplary forms of cerebral ischemic event, which may or may not be characterized as acquired or traumatic brain injuries, to which embodiments of the present disclosure relate.

The present disclosure contemplates the administration of inhibitors of the LIMK1 kinase. The inhibitor may affect LIMK1 expression and/or activity. The inhibitor may be a specific inhibitor of LIMK1 or may be selective for LIMK1. Thus, the inhibitor may also display inhibitory activity against LIMK2. The inhibitor may display inhibitory activity against one or more isoforms of LIMK1.

Contemplated herein are LIMK1 inhibitors in the form of, for example, small molecule inhibitors, nucleic acid-based (typically RNA-based) inhibitors such as RNAi, shRNA and ribozymes, peptide inhibitors and antibodies or antigen-binding fragments thereof. In particular exemplary embodiments of the present disclosure the inhibitor is a small molecule inhibitor. The skilled person will appreciate that the scope of the present disclosure is not to be limited by reference to any specific form or identity of LIMK1 inhibitor.

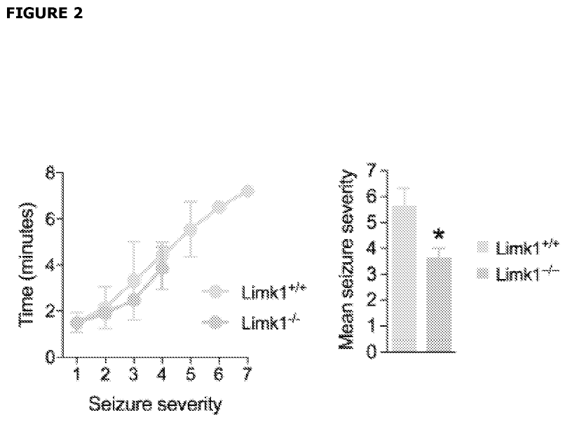

By way of example, LIMK1 inhibitors containing an aminothiazole scaffold are disclosed in Ross-Macdonald et al., 2008, Mol Cancer Ther 7:3490-3498 and in He et al, 2012, Bioorg Med Chem Lett 22:5995-5998). Other small molecules containing aminothienopyrimidines (see Sleebs et al., 2011, Bioorg Med Chem Lett 21:5992-5994 and Sleebs et al., 2011, Med Chem Commun 2:982-986), pyrrolopyrimidines (Manetti, 2018, Eur J Med Chem 155:445-448 and Harrison et al., 2009, J Med Chem 52:6515-6518) and other varying heterocyclic scaffolds (for example see FIG. 2 and Table 7 in Manetti, supra) have also been identified as LIMK1 inhibitors.

In a particular exemplary embodiment, the inhibitor comprises a compound of Formula (I) or a pharmaceutically acceptable salt thereof:

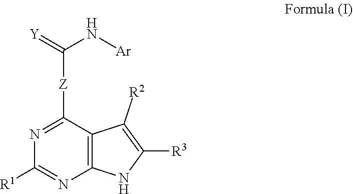

wherein:

Z is selected from the group consisting of optionally substituted cycloalkylene, optionally substituted arylene and optionally substituted aniline;

R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;

Y is selected from the group consisting of O, S, NCN, NCS and NSO₂Me;

Ar is selected from the group consisting of optionally substituted aryl, optionally substituted heteroaryl and optionally substituted heterocyclyl.

In some embodiments, Z is selected from one of the following structures:

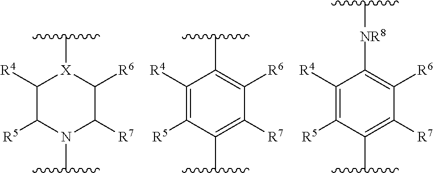

wherein:

R⁴, R⁵, R⁶ and R⁷ have the same definition as R² and R³ above;

X is CH or N; and

R⁸ is H or optionally substituted alkyl.

“Alkyl” refers to a monovalent alkyl groups that may be straight chained or branched, and preferably have from 1 to 10 carbon atoms, or more preferably 1 to 6 carbon atoms. Examples of such groups include methyl, ethyl, n-isopropyl, iso-propyl, n-butyl, iso-butyl, n-hexyl, and the like.

“Alkenyl” refers to a monovalent aliphatic carbocyclic group having at least one carbon-carbon double bond and which may be straight chained or branched, preferably having from 2 to 10 carbon atoms. Examples of such groups include a vinyl or ethenyl group (—CH═CH₂), n-propenyl (—CH₂CH═CH₂), iso-propenyl (—C(CH₃)═CH₂), but-2-enyl (—CH₂CH═CHCH₃), and the like.

“Alkynyl” refers to a monovalent aliphatic carbocyclic group having at least one carbon-carbon triple bond and which may be straight chained or branched, preferably having from 2 to 10 carbon atoms. Examples of such groups include an acetylene or ethynyl group (—C≡CH), propargyl (—CH₂C≡CH), and the like.

“Aryl” refers to a monovalent unsaturated aromatic carbocyclic group having a single ring (e.g. phenyl) or multiple condensed rings (e.g. naphthyl, anthracenyl), preferably having from 6 to 14 carbon atoms. Examples of aryl groups include phenyl, naphthyl, anthracenyl and the like.

“Alkoxy” and “aryloxy” refers to the groups “—O-alkyl” and “—O-aryl”, respectively, wherein the alkyl and aryl groups are described above.

“Halogen” refers to the groups fluoro, chloro, bromo and iodo.

“Heteroaryl” refers to a monovalent aromatic carbocyclic group, preferably having from 6 to 14 carbon atoms and 1 to 4 heteroatoms, wherein the heteroatoms are within the ring and are selected independently from oxygen, nitrogen and sulfur. Such heteroaryl groups can have a single ring (e.g. pyridyl, pyrrolyl or furyl) or multiple condensed rings (e.g. indolyl and benzofuryl).

“Heterocyclyl” refers to a monovalent saturated or unsaturated group having a single ring or multiple condensed rings, preferably having from 4 to 10 carbon atoms and from 1 to 4 heteroatoms, wherein the heteroatoms are selected independently from nitrogen, sulfur, oxygen, selenium and phosphorus.

Examples of heterocyclyl and heteroaryl groups include, but are not limited to pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo [b]thiophene, thiazole, thiazolidine, thiophene, benzo[b]thiophene, morpholino, piperidinyl, pyrrolidine, tetrahydrofuranyl, and the like.

As used herein, the term “optionally substituted” in relation to a particular group is taken to mean that the group may or may not be further substituted with one or more groups selected from hydroxyl, acyl, alkyl, alkoxy, alkenyl, alkenyloxy, alkynyl, alkynyloxy, amino, aminoacyl, alkylaryl, aryl, aryloxy, carboxyl, acylamino, cyano, halogen, nitro, sulphate, phosphate, phosphine, heteroaryl, heterocyclyl, oxyacyl, oxyacylamino, aminoacyloxy, trihalomethyl, and the like.

Examples of particularly suitable optional substituents include F, Cl, Br, I, CH₃, CH₂CH₃, OH, OCH₃, CF₃, OCF₃, NO₂, NH₂, COCH₃ and CN.

As used herein, the term “pharmaceutically acceptable salts” refers to salts that retain the desired biological activity of the parent compound, and include pharmaceutically acceptable acid addition salts and base addition salts. Suitable pharmaceutically acceptable acid addition salts of compounds of Formula (I) may be prepared from an inorganic acid or an organic acid. Examples of an inorganic acid include hydrochloric acid, sulphuric acid and phosphoric acid. Examples of organic acids include aliphatic, cycloaliphatic, aromatic, heterocyclic carboxylic and sulfonic organic acids, such as, formic, acetic, proprionic, succinic, glycolic, gluronic, lactic, malic, tartaric, citric, fumaric, maleic, alkylsulfonic and arylsulfonic acids. Where the compound of Formula (I) is a solid, the compounds and salts thereof may exist in one or more different crystalline or polymorphic forms, all of which are intended to be within the scope of Formula (I).

In a particular embodiment, the compound of Formula (I) is the compound of Formula (Ia):

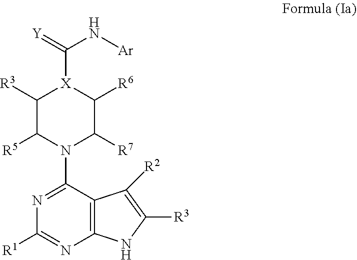

or a pharmaceutically acceptable salt thereof, wherein variables R¹ to R⁷, X, Y and Ar are as defined above.

In an embodiment, the compound of Formula (I) is the compound of Formula (Ia), or a pharmaceutically acceptable salt thereof, wherein:

R¹, R³, R⁵, R⁶ and R⁷ are H;

R² is methyl;

R³ is (S)-methyl;

X is N;

Y is NCN; and

Ar is 3-bromophenyl.

In an exemplary embodiment of the present disclosure, the compound of Formula (Ia) has the following structure (also referred to herein as LIMKi):

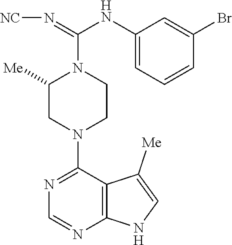 [SMILES: Cc1c[nH]c2ncnc(N3CCN(/C(=N\C#N)Nc4cccc(Br)c4)[C@@H](C)C3)c12]

Embodiments of the present disclosure contemplates the delivery of LIMK1 inhibitors to subjects in need of treatment by any suitable means, and typically in the form of pharmaceutical compositions, which compositions may comprise one or more pharmaceutically acceptable carriers, excipients or diluents. Such compositions may be administered in any convenient or suitable route such as by parenteral (e.g. intraperitoneal, subcutaneous, intraarterial, intravenous, intramuscular, intrathecal, intracerebral, intraocular), oral (including sublingual), nasal, transmucosal or topical routes. In circumstances where it is required that appropriate concentrations of the molecules are delivered directly to the site in the body to be treated, administration may be regional rather than systemic. Regional administration provides the capability of delivering very high local concentrations of the molecules to the required site and thus is suitable for achieving the desired therapeutic or preventative effect whilst avoiding exposure of other organs of the body to the vectors and molecules and thereby potentially reducing side effects.

As will be appreciated by those skilled in the art, the choice of pharmaceutically acceptable carrier or diluent will be dependent on the route of administration and on the nature of the condition and subject to be treated. The particular carrier or diluent and route of administration may be readily determined by a person skilled in the art. The carrier or diluent and route of administration should be carefully selected to ensure that the activity of the compound is not depleted during preparation of the formulation and the compound is able to reach the site of action intact.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and me thylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

A person skilled in the art will readily be able to determine appropriate formulations for the compound to be administered using conventional approaches. Techniques for formulation and administration may be found in, for example, Remington (1980) Remington's Pharmaceutical Sciences, Mack Publishing Co., Easton, Pa., latest edition; and Niazi (2009) Handbook of Pharmaceutical Manufacturing Formulations, Informa Healthcare, New York, second edition, the entire contents of which are incorporated by reference.

Identification of preferred pH ranges (where appropriate) and suitable excipients is routine in the art, for example, as described in Katdare and Chaubel (2006) Excipient Development for Pharmaceutical, Biotechnology and Drug Delivery Systems (CRC Press).

In some embodiments, a suitable compound or agent is formulated for oral administration in a dosage form such as a tablet, pill, capsule, liquid, gel, syrup, slurry, suspension, lozenge and the like for oral ingestion by a subject. In particular embodiments, the compound or agent is formulated for oral administration in a solid dosage form, such as a tablet, pill, lozenge or capsule. In such embodiments, the pharmaceutically acceptable carrier may comprise a number of excipients including, but not limited to, a diluent, disintegrant, binder, lubricant, and the like.

Suitable diluents (also referred to as “fillers”) include, but are not limited to, lactose (including lactose monohydrate, spray-dried monohydrate, anhydrous, etc.), mannitol, xylitol, dextrose, sucrose, sorbitol, compressible sugar, isomalt, microcrystalline cellulose, powdered cellulose, starch, pregelatinised starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose, silicates (e.g. silicon dioxide), polyvinyl alcohol, talc, and combinations thereof.

Suitable disintegrants include, but are not limited to, sodium carboxymethyl cellulose, pregelatinised starch, calcium carboxymethyl cellulose, croscarmellose sodium, crospovidone, polyvinylpyrrolidone, methylcellulose, sodium starch glycolate, microcrystalline cellulose, lower alkyl-substituted hydroxypropyl cellulose, starch, sodium alginate and combinations thereof Suitable binders include, but are not limited to, microcrystalline cellulose, gelatine, sugars, polyethylene glycol, natural and synthetic gums, polyvinylpyrrolidone, pregelatinized starch, hydroxypropyl cellulose, hydroxypropyl methylcellulose and combinations thereof. Suitable lubricants include, but are not limited to, magnesium stearate, calcium stearate, zinc stearate, sodium stearyl fumarate, polyethylene glycol and combinations thereof.

Pharmaceutical formulations for parenteral administration include aqueous solutions of a suitable compound or agent in water-soluble form. Additionally, suspensions of the compound or agent may be prepared as appropriate oily injection suspensions. Suitable lipophilic solvents or carriers include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilisers or agents that increase the solubility of the compounds to allow for the preparation of highly concentrated solutions.

Sterile solutions may be prepared by combining the compound or agent in the required amount in the appropriate solvent with other excipients as described above as required, followed by sterilization, such as filtration. Generally, dispersions are prepared by incorporating the various sterilised active compounds into a sterile vehicle which contains the basic dispersion medium and the required excipients as described above. Sterile dry powders may be prepared by vacuum- or freeze-drying a sterile solution comprising the active compounds and other required excipients as described above.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination. For injection, compositions of the invention may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

It will be understood that the specific dose level of a composition of the invention for any particular subject will depend upon a variety of factors including, for example, the activity of the inhibitor employed, the half-life of the inhibitor, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

In exemplary embodiments of the present disclosure it is contemplated that the inhibitor may be administered to a subject daily or less than daily, for example every second day or every third day for the duration of treatment required to achieve the desired outcome. Administration may be continuous, for example on a daily basis or every second day, or may be intermittent with spacing between administrations determined by the treating medical professional depending on response of the subject to treatment and progress of the subject during the course of treatment.

The present invention contemplates combination therapies, wherein LIMK1 inhibitors as described herein are coadministered with other suitable agents that may facilitate the desired therapeutic or prophylactic outcome. By “coadministered” is meant simultaneous administration in the same formulation or in two different formulations via the same or different routes or sequential administration by the same or different routes. By “simultaneously” is meant that the active agents are administered at substantially the same time. By “sequential” administration is meant a time difference of from seconds, minutes, hours or days between the administration of the agents. Administration may be in any order.

Each embodiment described herein is to be applied mutatis mutandis to each and every embodiment unless specifically stated otherwise.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the disclosure.

### EXAMPLES

The following examples are illustrative of the disclosure and should not be construed as limiting in any way the general nature of the disclosure of the description throughout this specification.

### General Methods

Mice. APP23 express human Swedish mutant (K595N/M596L) amyloid-β precursor protein (APP) in neurons (Sturchler-Pierrat et al., 1997, Proc Natl Acad Sci USA 94, 13287-13292). Limk1^{-/-} mice have been reported previously (Meng et al., 2002, Neuron 35, 121-133). All mice were on a pure C57Bl/6 background. Mice were housed in standard individually ventilated cages on a 12 hour light/dark cycle with access to standard chow and water ad libitum. Mice of both genders were used unless otherwise indicated. Investigators were blinded to genotypes until after completion of data analysis. All experiments were approved by the Animal Ethics Committee of Macquarie University.

Electroencephalography. Hippocampal EEG recordings were done with implanted telemetric electrodes (D.S.I.) as previously described (Ittner et al., 2014, Acta neuropathologica communications 2, 149). All recordings were done with mice individually housed in their home cages one week after implantation of transmitters. Compound administration was done intraperitoneally during transient restraining of mice. Vehicle for the LIMK1 inhibitor LIMKi contained hydroxypropyl methylcellulose 606 (HPMC 606) with polyvinylpyrrolidone k17 (PVP k17) and tween 80 in a proportion of 0.5%, 0.5%, 0.1%, respectively. Spike analysis was done with the Neuroscore software module (D.S.I.) as previously described (Ittner et al., 2016, Science 354, 904-908). Cross frequency coupling of theta and gamma waves was performed as previously described (Ittner et al., 2016, Science 354, 904-908).

Seizure model. Excitotoxic seizure were induced in mice by intraperitoneal injection of 50 mg/kg body weight pentylenetetrazole followed by observation in a square area (40×40 cm). Scoring of seizures was done as previously described (Ittner et al., 2010, Cell 142, 387-397). For LIMKi-treated mice, scoring was adjusted by combining the severe seizure scores 5 to 7 into a single score of 5. For both scales, minor to moderate seizures are reflected by scores <5.

Statistical analysis. All statistical analyses were done with the Prism 7 software (GraphPad). All values are presented as mean and standard error of the mean. P values <0.05 were considered significant.

### Example 1

### Limk1 Depletion in APP23 Transgenic Mice

Mouse models with transgenic expression of human mutant APP present with memory deficits, Aβ pathology and premature mortality, which has been associated with excitotoxicity (Ittner and Gotz, 2011, Nat Rev Neurosci 12:67-72).

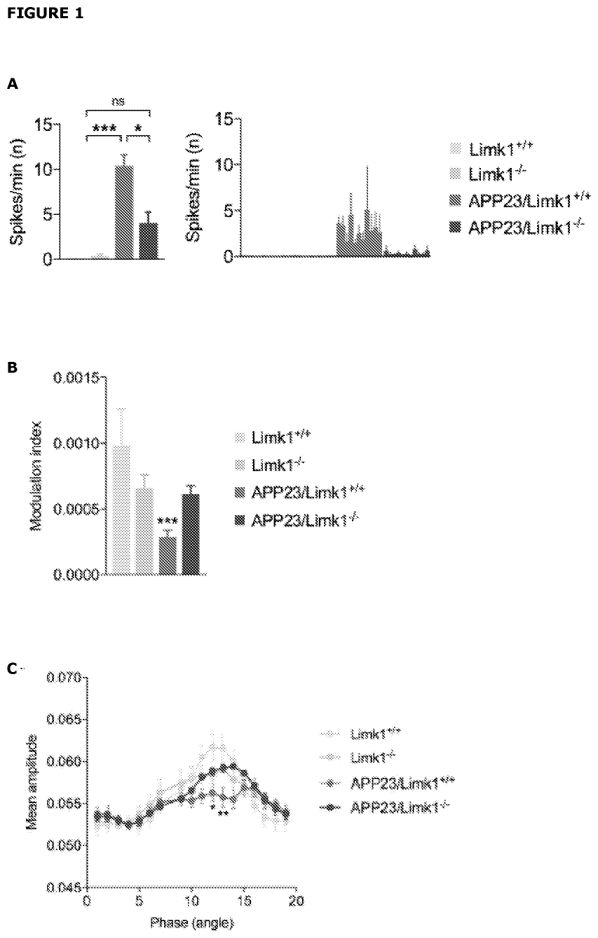

APP23 mice present with non-convulsive, silent seizure activity during electroencephalography (EEG) recordings, as well as disrupted cross frequency coupling (CFC) of θ phase modulation of γ power during no-spike episodes of EEG recordings (Ittner et al., 2014, Acta Neuropath Comm 2:149), a modality linked to memory formation including in humans. The inventors implanted APP23/Limk1^{-/-} mice, as well as APP23/Limk1^{+/+}, Limk1^{-/31} and Limk1^{+/+} littermates with telemetric EEG transmitters off hippocampal electrodes for recording in freely moving mice. APP23/Limk1^{+/+} mice presented with frequent hypersynchronous discharges during EEG recordings, while there were virtually no such events detected in Limk1^{-/-} and Limk1^{+/+} littermate recordings (FIG. 1A). For comparison, APP23/Limk1^{-/-} mice showed significantly reduced numbers of spikes that were not significantly different from controls. CFC during spike-free episodes was disrupted in APP23/Limk1+/+ mice (data not shown). In contrast, APP23/Limk1^{-/-} mice showed the same CFC at 8Hz as detected in Limk1^{-/-} and Limk1^{+/+} littermate recordings. Accordingly, the significantly reduced modulation index (FIG. 1B) and amplitude phase (FIG. 1C) of APP23/Limk1+/+ mice was normalized in APP23/Limk1^{-/-} mice to levels of Limk1^{-/-} and Limk1^{+/+} littermate recordings Thus, neuronal network aberrations of APP23 mice were mitigated by knocking out Limk1.

### Example 2

### Limk1^{-/-} Mice are Less Susceptible to Excitotoxic Seizure

Excitotoxicity contributes to deficits in APP transgenic mice (see, e.g., Ittner et al., Science 354:904-908). The inventors tested whether Limk1 depletion could protect mice from induced seizures. When challenged with 50 mg/kg pentylenetetrazol (PTZ), Limk1^{-/-} mice developed significantly less severe seizures than Limk1^{+/+} littermates (FIG. 2). Latency to develop low grade symptoms upon PTZ administration were comparable in Limk1^{-/-} and Limk1^{+/+} mice, but only Limk1^{+/+} mice progressed to convulsive seizure and status epilepticus (FIG. 2). Hence, Limk1 mediates progression to convulsive seizures in a mouse model of epilepsy.

### Example 3

### Pharmacological LIMK1 Inhibition Reduces Excitotoxic Seizures

Given the improvements demonstrated in APP23/Limk1^{-/-} mice (Example 2), the inventors determined whether treatment with a LIMK1 inhibitor could similarly reduce excitotoxic seizures in APP23 mice. For this purpose Compound 1, shown below (also referred to herein as LIMKi), was used.

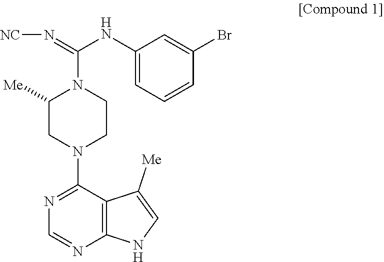 [SMILES: Cc1c[nH]c2ncnc(N3CCN(/C(=N\C#N)Nc4cccc(Br)c4)[C@@H](C)C3)c12]

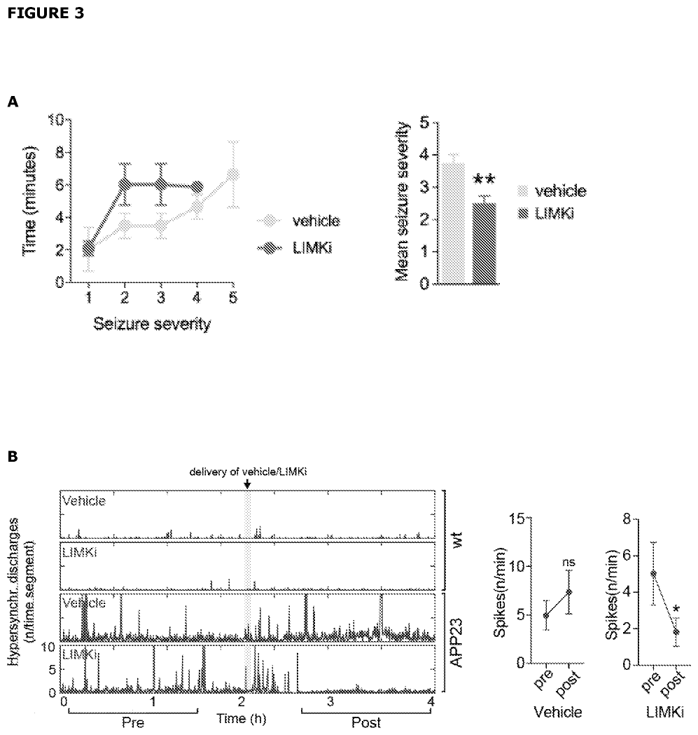

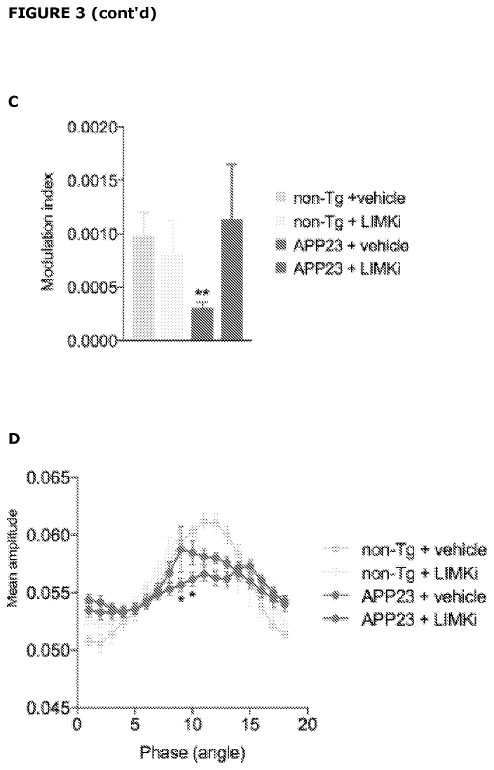

Administration of 10 mg/kg Compound 1 30 minutes prior to inducing excitotoxic seizures with PTZ, significantly reduced mean seizure severity and increased the latency to develop severe seizures in C57Bl/6 mice (FIG. 3A). Next, the inventors tested the effects of acute Compound 1 administration on neuronal network activity in APP23 mice. Recordings prior to drug administration showed frequent hypersynchronous discharges in APP23 mice (FIG. 3B). Similarly, continued recordings over 2 hours after vehicle injections showed high spike activity. However, 30 minutes after Compound 1 administration, hypersynchronicity significantly decreased in Compound 1 treated APP23 mice compared to vehicle-treated APP23 mice and compared to recordings prior to injections (FIG. 3B). Furthermore, CFC 1 hour after Compound 1 administration was re-established (data not shown). The significantly reduced modulation index (FIG. 3C) and amplitude phase (FIG. 3D) was normalized by Compound 1 administration in APP23 mice. Thus, inhibition of LIMK1 with Compound 1 conferred protection from Aβ toxicity in neurons and from excitotoxicity in vivo. Furthermore, treatment of APP23 with Compound 1 corrected network aberrations including CFC.

1. A method for treating or preventing an excitotoxicity-related condition in a subject, the method comprising administering to the subject an effective amount of an inhibitor of Lim-domain kinase 1 (LIMK1).

2-26. (canceled)

27. The method according to claim 1, wherein the excitotoxicity-related condition is associated with seizures.

28. The method according to claim 27, wherein the seizures are absence seizures, tonic seizures, atonic seizures, clonic seizures, myoclonic seizures or tonic-clonic seizures.

29. The method according to claim 1, wherein the excitotoxicity-related condition results from or is associated with a cerebral ischemic event.

30. The method according to claim 29, wherein the cerebral ischemic event comprises a traumatic brain injury or stroke.

31. The method according to claim 1, wherein the excitotoxicity-related condition is epilepsy.

32. The method according to claim 1, wherein treating or preventing the excitotoxicity-related condition comprises reducing the severity of a seizure or of seizures over time, increasing the latency to develop a seizure or seizures over time, and/or reducing the frequency of seizures.

33. The method according to claim 1, wherein treating or preventing the excitotoxicity-related condition comprises reducing excitotoxicity in neurons and/or for protecting neurons from excitotoxicity.

34. A method for treating or preventing seizures in a subject in need thereof, the method comprising administering to the subject an effective amount of an inhibitor of LIMK1.

35. The method according to claim 34, wherein the seizures are absence seizures, tonic seizures, atonic seizures, clonic seizures, myoclonic seizures or tonic-clonic seizures.

36. The method according to claim 34, wherein treating or preventing the seizures comprises reducing the severity of a seizure or of seizures over time, increasing the latency to develop a seizure or seizures over time, and/or reducing the frequency of seizures.

37. The method according to claim 34, wherein treating or preventing the seizures comprises protecting neurons from excitotoxicity.

38. A method for reducing excitotoxicity in neurons and/or protecting neurons from excitotoxicity, the method comprising exposing neurons to an effective amount of an inhibitor of LIMK1.

39. The method according to claim 1, wherein the inhibitor of LIMK1 comprises a compound of Formula (I) or a pharmaceutically acceptable salt thereof: 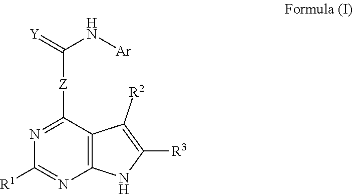
wherein:
Z is selected from the group consisting of optionally substituted cycloalkylene, optionally substituted arylene and optionally substituted aniline;
R¹, R² and R³ are independently selected from the group consisting of H, halogen, nitro, cyano, hydroxyl, optionally substituted alkoxy, optionally substituted amine, optionally substituted alkyl, optionally substituted heteroalkyl and optionally substituted alkenyl;
Y is selected from the group consisting of O, S, NCN, NCS and NSO₂Me; and
Ar is selected from the group consisting of optionally substituted aryl, optionally substituted heteroaryl and optionally substituted heterocyclyl.

40. The method according to claim 39, wherein Z is selected from one of the following structures: 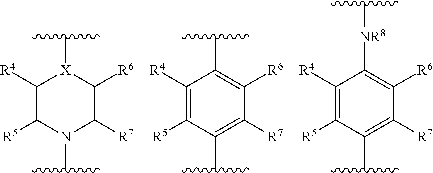
wherein:
R⁴, R⁵, R⁶ and R⁷ have the same definition as R¹, R² and R³ above;
X is CH or N; and
R⁸ is H or optionally substituted alkyl.

41. The method according to claim 39, wherein the compound of Formula (I) is the compound of Formula (Ia) or a pharmaceutically acceptable salt thereof: 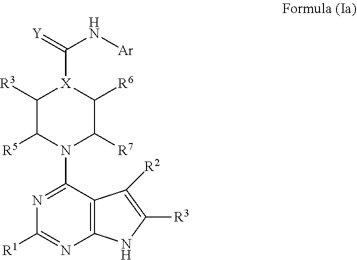
wherein variables R¹ to R⁷, X, Y and Ar are as defined in claim 1 or 2.

42. The method according to claim 39, wherein the compound of Formula (I) is the compound of Formula (Ia), or a pharmaceutically acceptable salt thereof, wherein:
R¹, R³, R⁵, R⁶ and R⁷ are H;
R² is methyl;
R³ is (S)-methyl;
X is N;
Y is NCN; and
Ar is 3-bromophenyl.

43. The method according to claim 39, wherein the compound of Formula (I) has the following structure:

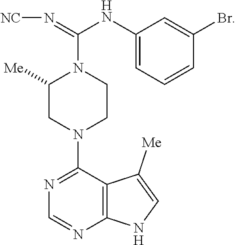 [SMILES: Cc1c[nH]c2ncnc(N3CCN(/C(=N\C#N)Nc4cccc(Br)c4)[C@@H](C)C3)c12]

44. The method according to claim 1, wherein the inhibitor is a selective inhibitor of LIMK1.

45. The method according to claim 1, wherein the inhibitor is a specific inhibitor of LIMK1.
